(19) United States
(12) Patent Application Publication
(10) Pub. No.: US 2015/0376549 A1
(43) Pub. Date: Dec. 31, 2015

(54) TREATMENT OF HARD SURFACES

(71) Applicant: CRODA, INC., Edison, NJ (US)

(72) Inventors: JOSEPH WILLIAM SCHEBLEIN, FLEMINGTON, NJ (US); JEFFREY PATRICK WU, EAST BRUNSWICK, NJ (US); ANDREW JAMES KAZISKA, WHITEHOUSE STATION, NJ (US)

(73) Assignee: CRODA, INC., EDISON, NJ (US)

(21) Appl. No.: 14/769,178

(22) PCT Filed: Feb. 6, 2014
(86) PCT No.: PCT/US2014/015065

§ 371 Date: Aug. 20, 2015

Related U.S. Application Data
(60) Provisional application No. 61/768,013, filed on Feb. 22, 2013.

(51) Int. Cl. C11D 3/00; C07D 307/20; C11D 3/20

(52) U.S. Cl. CPC C11D 3/0036; C11D 3/2093; C07D 307/20

(57) ABSTRACT

Cleaning compositions are disclosed herein. The cleaning compositions comprises a reaction product of an alkoxylated polyol or ester thereof, and a dicarboxylic acid. Methods of cleaning hard surfaces using the cleaning compositions and formulations of the cleaning compositions are also disclosed. Methods of treating hard surfaces to improve soil resistance of a hard surface are also disclosed.

This application is related to and claims the benefit of U.S. Provisional Application No. 61/768,013 entitled “TREATMENT OF HARD SURFACES” filed on Feb. 22, 2013, the contents of which are incorporated herein by reference.

### FIELD OF THE INVENTION

The present invention relates to novel cleaning compositions comprising surface modifier compounds, and in particular, use of said surface modifiers in the treatment of hard surfaces and to methods of treating hard surfaces to provide soil resistance.

### BACKGROUND OF THE INVENTION

It is common experience that hydrophobic soils can be difficult to remove from hard surfaces. In comparison, hydrophilic soils are usually easier to remove with aqueous wash systems. It is therefore particularly useful to treat hard surfaces to improve their resistance to said hydrophobic soils. A further benefit of improving the soil resistance of hard surfaces is that it may reduce the tendency to form water tide marks and/or the tendency to leave streaks especially after rinsing.

An improvement in the soil repellency of hard surfaces is important in that it reduces the tendency of soil material to adhere to the surfaces, thereby slowing the rate or reducing the extent of soiling. Additionally, the improved soil repellency can make it easier to remove the soil when cleaning the surface by reducing the level of mechanical effort required.

Previous attempts to provide soil resistance include the use of formulations including copolymers of acrylic esters/amides carrying quaternium, particularly diquaternium, substituents for example as described in U.S. Pat. No. 6,703,358. Other hard surface treatments, in particular for oily soils, are described in WO 2011/051646 which discloses use of polymeric compounds to treat surfaces. US 2011/0039029 discloses treatment compositions and systems for forming a detachable coating on a surface for easier cleaning. US 2008/0250978 discloses use of hydrophobic nanoparticles in a water based, low volatile organic compound super hydrophobic coating composition that can be used to make wet and dry dirt repellent surfaces to keep the surfaces clean.

The present invention therefore seeks to provide a composition which may provide soil resistance for hard surfaces, and in particular which provides improved soil resistance to a hard surface to which it is applied in comparison with an un-treated surface. The present invention further seeks to provide a method of treating a hard surface with said composition having said soil resistance properties.

### SUMMARY OF THE INVENTION

According to a first aspect of the present invention there is provided a cleaning composition comprising the reaction product of:
a) an alkoxylated polyol or ester thereof; and
b) a dicarboxylic acid.

According to a second aspect of the present invention there is provided a method of treating a hard surface which comprises applying to the surface a cleaning composition, said composition comprising the reaction product of:
a) an alkoxylated polyol or ester thereof; and
b) a dicarboxylic acid.

According to a third aspect of the present invention there is provided a cleaning formulation, said formulation comprising in the range from 0.01 wt. % to 40 wt. % of the cleaning composition of the first aspect.

In particular, the cleaning composition of the first aspect, the method of the second aspect, and the cleaning formulation of the third aspect are suitable for use in providing soil resistance to a hard surface.

According to a fourth aspect of the present invention there is provided the use of a cleaning composition, according to the first aspect or a cleaning formulation according to the third aspect, for providing soil resistance to a hard surface.

### DETAILED DESCRIPTION OF THE INVENTION

It has been found that the reaction product of an alkoxylated polyol/ester and a dicarboxylic acid provides a cleaning composition which, when applied to a hard surface, enhances both soil repellency and soil release properties. The composition has been found to give good resistance to the deposition of soils. It has also been found that the combination of the present application can provide soil repellency and release when added to end-use cleaning formulations with a wide pH range.

Without wishing to be bound by theory, it has been found that the benefits of the invention may be conferred by the cleaning composition providing a relatively hydrophilic coating which imparts or improves soil resistance to the surface to which it is applied.

As used herein, the terms ‘for example,’ for instance,′ such as,′ or ‘including’ are meant to introduce examples that further clarify more general subject matter. Unless otherwise specified, these examples are provided only as an aid for understanding the applications illustrated in the present disclosure, and are not meant to be limiting in any fashion.

It will be understood that, when describing the number of carbon atoms in a substituent group (e.g. ‘C₁ to C₆ alkyl’), the number refers to the total number of carbon atoms present in the substituent group, including any carbon atoms present in any branched groups. Additionally, when describing the number of carbon atoms in, for example, fatty acids, this refers to the total number of carbon atoms including the one at the carboxylic acid, and any carbon atoms present in any branch groups.

It will be understood that use of the term ‘soil resistance’ as used in the present application should be understood to refer to soil repellency and/or soil release properties. Therefore, when describing the invention of the present application as having or improving soil resistance, this should be understood as the invention providing or imparting improved soil repellence to prevent build-up of soils on a surface, and/or soil release properties to surfaces to facilitate subsequent cleaning.

In the context of this invention ‘soil resistance’ is imparting improved soil repellency and/or soil release properties to surfaces when compared to a non-treated surface, for example, in order to facilitate subsequent cleaning. In at least one embodiment, the non-treated surface refers to hard surfaces, notably hard surfaces in domestic and industrial and/or institutional cleaning (often abbreviated to “I & I Cleaning”).

It will be understood that use of the term ‘hard surface(s)’ as used in the present application should be understood as referring to solid surfaces, particularly but not exclusively to substantially non-porous surfaces such as those of metals, ceramics, glass, wood, and plastics, particularly laminated plastics, all including painted, varnished, or sealed surfaces.

Examples of hard surfaces include: walls, floors, windows, minors, doors, tiles and tiled areas, work surfaces, including cutting and chopping boards, domestic fittings e.g. shelves and cupboards, washing and sanitary fixings, e.g., sinks, wash basins, baths, showers and WCs, domestic appliances, e.g., stoves, ovens, including microwave ovens, washing machines and dryers, dishwashers, refrigerators, freezers and chillers, food preparation machines, e.g., mixers, blenders and food processors, in both domestic and institutional and industrial environments, including in hospitals, medical laboratories and medical treatment environments.

When used herein, the term ‘dicarboxylic’ refers to an acid comprising two functional groups, i.e., carboxylate groups. The term dicarboxylic acid defines the group of compounds containing both bi-functional carboxylic acids and dimer acids.

The polyol in the alkoxylated species preferably comprises at least 3 hydroxyl groups. Preferably, the polyol in the alkoxylated species comprises up to 9 hydroxyl groups. Desirably, the polyol has an average of 1 or 2 primary hydroxyl groups and at least 1, preferably 1 to 4 secondary hydroxyl groups.

Preferably, the polyol in the alkoxylated species has the formula (I):

R¹—(OH)ₙ (I)

where n is from 3 to 8, and particularly from 3 to 6.

The group R¹ is desirably an aliphatic hydrocarbyl group. Preferably, the group R¹ is saturated. Preferably, R¹ has from 3 to 10 carbon atoms, preferably from 3 to 8, and especially from 3 to 6 carbon atoms. R¹ will usually be linear, though it may include branching. Desirably, the polyol in the alkoxylated species has the general formula (Ia):

HOH₂C—(CHOH)ₚ—CH₂OH (Ia)

where p is from 1 to 6, more preferably from 1 to 4.

Suitable polyols include glycerol, C₄ polyols such as threitol and erythritol, C₅ polyols such as inositol, arabitol and xylitol, and C₆ polyols such as sorbitol, and compounds derived therefrom, for example sorbitan. The C₄ to C₆ polyols are commonly the reduced or hydrogenated forms of corresponding tetrose, pentose and hexose sugars. Desirably the polyol is glycerol or a derivative thereof, particularly sorbitol or sorbitan (usually derived in situ from sorbitol) or a mixture or combination of these.

The polyol may be present in an esterified form. Preferably, when the polyol is sorbitan, the sorbitan is present in the form of an ester derived from the reaction of the sorbitan with a fatty acid or derivative thereof. Preferred fatty acids or derivatives thereof comprise in the range from 6 to 24, more preferably 8 to 20, particularly 10 to 18, and especially 12 to 16 carbon atoms. Linear fatty acids are preferred. Suitable fatty acids include capric, lauric, myristic, palmitic, stearic, and/or behenic acid.

Suitable fatty acids or derivatives thereof for reaction with the sorbitol or derivative thereof are preferably derived from natural sources, preferably from vegetable sources. For example, lauric acid is the main acid in coconut oil and in palm kernel oil. It may also be found in animal milk, for example cow's milk and goat's milk. The fatty acids or derivatives thereof may be derived from palm oil, American palm oil, nutmeg oil, peach palm seed oil, betel nut, date seed, macadamia nut oil, watermelon seed oil, pumpkin seed or flower oil, and other vegetable sources.

In one embodiment, the polyol or ester thereof is a sorbitan compound, more specifically, a sorbitan ester. Suitable sorbitan esters include sorbitan cocoate, sorbitan caprate, sorbitan laurate, sorbitan myristate, sorbitan palmitate, and/or sorbitan stearate. Preferred sorbitan esters are sorbitan caprate and/or sorbitan laurate, more preferably sorbitan monolaurate.

The polyol present is alkoxylated. The alkoxylated polyol preferably comprises residues of an alkoxy group, preferably a univalent radical R²—O—, or anion R²—O⁻, where R² is an alkyl group, preferably a C₁ to C₆ alkyl group, preferably C₁ to C₄ and more preferably C₂ to C₃.

Preferably, the alkoxy group is a methoxy group, ethoxy group, or propoxy group. More preferably, the alkoxy group is an ethoxy or propoxy group.

The presence of ethoxy groups in the alkoxylated species may increase the hydrophilic-lipophilic balance (HLB) of the alkoxylated species. The presence of propoxy groups in the alkoxylated species may lower the HLB of the alkoxylated species.

Preferably, ethoxy groups are present in the alkoxylated polyol or ester thereof at a pre-determined concentration to provide the desired water solubility and/or HLB. Alternatively, a mixture of ethoxy and propoxy groups may be present to provide the desired water solubility and/or HLB.

Preferably, the alkoxylated polyol or ester thereof is derived from the reaction of an alkylene oxide with the polyol or ester thereof. One or more equivalents of alkylene oxide may react with each polyol molecule or molecule of the ester thereof. Preferably, the polyol is polyalkoxylated. Preferably, the alkylene oxide is selected from the group comprising C₁ to C₆ alkylene oxides, preferably C₁ to C₄ and more preferably C₂ to C₃ alkylene oxides. Preferably, the alkylene oxide is ethylene oxide, or propylene oxide, or a mixture thereof.

Preferably, the alkoxylated polyol or ester thereof comprises between 1 and 500 alkylene oxide equivalents per molecule, more preferably between 1 and 400, further preferably between 1 and 200, and most preferably between 2 and 100 alkylene oxide equivalents per molecule.

Where the number of equivalents of alkylene oxide is given in terms of per molecule, preferably, this is the average number of equivalents per molecule in a sample of the product. Individual molecules in the sample may have fewer or greater than the stated number of equivalents of alkylene oxide, but on average, the sample will comprise molecules having an average of the stated number of equivalents of alkylene oxide.

Where the cleaning composition comprises an alkoxylated polyol, there are preferably between 1 and 500 alkylene oxide equivalents per molecule, more preferably between 2 and 400, further preferably between 5 and 200, and most preferably between 10 and 100 alkylene oxide equivalents per polyol molecule.

Where the cleaning composition comprises an alkoxylated ester of a polyol, there are preferably between 1 and 500 alkylene oxide equivalents per molecule. More preferably, between 2 and 300, further preferably between 3 and 150, and most preferably between 5 and 50 alkylene oxide equivalents per molecule.

In one embodiment, the alkoxylated polyol is preferably an alkoxylated sorbitol, more preferably an ethoxylated sorbitol. Preferably, the alkoxylated polyol comprises between 1 and 500 alkylene oxide equivalents per molecule, preferably between 1 and 400, more preferably between 1 and 200, and most preferably between 2 and 100 alkylene oxide equivalents per sorbitol molecule. Preferably, the alkoxylated sorbitol has the general structure (II):

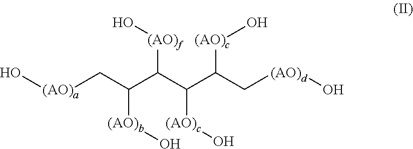

wherein;
a, b, c, d, e, and f may each independently be any number between 0 and 100;
AO is an alkylene oxide residue, preferably an ethylene oxide (EO) residue; and
where the sum of a, b, c, d, e, and f is between 1 and 500, preferably between 1 and 400, more preferably between 1 and 200, even more preferably between 2 and 100, and most preferably between 10 and 40.

In at least one embodiment, the alkoxylated polyol is an ethoxylated sorbitol, more preferably a polyoxyethylene (X) sorbitol, wherein X is a number between 1 and 40. More preferably, the alkoxylated polyol is polyoxyethylene (10) sorbitol or polyoxyethylene (40) sorbitol, where the sum of a, b, c, d, e, and f in formula (II) is 10 or 40. Most preferably, the alkoxylated polyol is polyoxyethylene (40) sorbitol, where the sum of a, b, c, d, e, and f in formula (II) is 40. Polyoxyethylene (40) sorbitol is available commercially from Croda under the trade name Atlas™ G2004.

In another embodiment, the alkoxylated polyol ester is preferably an alkoxylated sorbitan ester, more preferably an ethoxylated sorbitan ester. Preferably, the alkoxylated polyol ester comprises between 1 and 500 alkylene oxide equivalents per molecule, preferably between 1 and 400, more preferably between 1 and 200, and most preferably between 2 and 100 alkylene oxide equivalents per sorbitan ester molecule. Preferably, the alkoxylated sorbitan ester has the general structure (III):

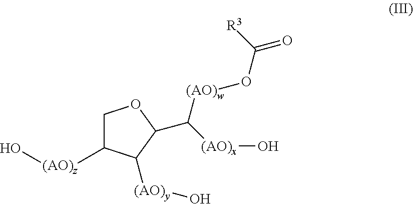

wherein
w, x, y, and z may each independently be any number between 0 and 100;
AO is an alkylene oxide residue, preferably an ethylene oxide (EO) residue;
R³ is an alkyl group; and
where the sum of w, x, y, and z is between 1 and 300, preferably between 2 and 200, more preferably between 3 and 100 and most preferably between 5 and 50.

In formula (III), R³ may be saturated or unsaturated, preferably saturated. R³ preferably comprises between 1 and 29 carbon atoms, more preferably between 5 and 25, further preferably between 9 and 21, most preferably between 11 and 17. Preferably R³ is derived from a fatty acid, more preferably selected from the group comprising lauric acid, palmitic acid, stearic acid, and oleic acid.

Preferably, in this embodiment, the alkoxylated polyol ester is an ethoxylated sorbitan ester. More preferably, an ethoxylated sorbitan monolaurate, monopalmitate, monostearate, or monooleate. Further preferably, an ethoxylated sorbitan monolaurate. Most preferably, polyoxyethylene (20) sorbitan monolaurate, where the sum of w, x, y, and z in formula (III) is 20. Polyoxyethylene (20) sorbitan monolaurate is available commercially from Croda under the trade name Tween™ 20.

The dicarboxylic acid present preferably has from 4 to 40 carbon atoms. Preferably, the dicarboxylic acid is aliphatic. Typically, the dicarboxylic acid is of the formula (IV):

HOOC—R⁴—COOH (IV)

where R⁴ is a C₂ to C₃₆ hydrocarbyl group which can be saturated or unsaturated, linear or branched and can be aromatic, e.g., a phenyl ring (thus giving a phthalic, terephthalic or iso-phthalic dicarboxylic acid) or, desirably, aliphatic, e.g., an alkylene or alkenylene group, and may be cyclic though it is desirably open chain. Commonly R⁴ is a group: —(CH₂)ₘ—, where m is from 2 to 36. Suitable reactive derivatives of the dicarboxylic acids include lower (e.g., C₁ to C₄ and particularly methyl) alkyl esters (usually diesters) and anhydrides, particularly cyclic anhydrides such as succinic, maleic and phthalic anhydrides.

In one embodiment, the dicarboxylic acid has at least 4 carbon atoms, preferably at least 5 and more preferably at least 6 carbon atoms. In this embodiment, the dicarboxylic acid preferably comprises up to 36 carbon atoms, preferably up to 20 carbon atoms, more preferably up to 12 carbon atoms and most preferably up to 10 carbon atoms. In this embodiment, the dicarboxylic acid may be selected from the group comprising malonic acid, succinic acid, glutaric acid, adipic acid, pimelic acid, suberic acid, axelaic, acid and sebacic acid. Preferably, the dicarboxylic acid may be selected from adipic acid, suberic acid, and sebacic acid. More preferably, the dicarboxylic acid is adipic acid.

In another embodiment, the dicarboxylic acid is preferably a dimer acid. In this embodiment, the dimer acid preferably comprises from 24 to 52 carbon atoms, preferably from 28 to 48 carbon atoms, more preferably from 32 to 46 carbon atoms and most preferably from 36 to 44 carbon atoms. Preferably the dimer acid is a C₃₆ dimer acid.

The term dimer fatty acid is well known in the art and refers to the dimerisation product of mono- or polyunsaturated fatty acids and/or esters thereof. Preferred dimer acids are dimers of C₁₀ to C₃₀, more preferably C₁₂ to C₂₄, particularly C₁₄ to C₂₂, and especially C₁₈ alkyl chains. Suitable dimer fatty acids include the dimerisation products of oleic acid, linoleic acid, linolenic acid, palmitoleic acid, and elaidic acid. The dimerisation products of the unsaturated fatty acid mixtures obtained in the hydrolysis of natural fats and oils, e.g., sunflower oil, soybean oil, olive oil, rapeseed oil, cottonseed oil and tall oil, may also be used. Hydrogenated dimer fatty acids, for example by using a nickel catalyst, may also be employed.

In addition to the dimer fatty acids, dimerisation usually results in varying amounts of oligomeric fatty acids (so-called “trimer”) and residues of monomeric fatty acids (so-called “monomer”), or esters thereof, being present. The amount of monomer can, for example, be reduced by distillation. Particularly preferred dimer fatty acids have a dicarboxylic (or dimer) content of greater than 70%, more preferably greater than 85%, and particularly greater than 94% by weight.

Preferably, the molar ratio of alkoxylated polyol or ester thereof to dicarboxylic acid in the reaction product is at least 0.05:1, preferably at least 0.1:1, more preferably at least 0.5:1, and most preferably at least 1:1. Preferably, the molar ratio of alkoxylated polyol or ester thereof to dicarboxylic acid in the reaction product is up to 20:1, preferably up to 10:1, more preferably up to 5:1, and most preferably up to 3:1.

Optionally, the reaction product of the alkoxylated polyol or ester thereof and the dicarboxylic acid may further comprise an end-cap. Preferably, the end-cap comprises a monovalent radical. Preferably, the end-cap comprises a monocarboxylic acid.

Preferably, the monocarboxylic acid is a fatty acid. Preferably, the monocarboxylic acid comprises from 2 to 30 carbon atoms, more preferably between 12 and 26, further preferably between 14 and 22 carbon atoms, and most preferably between 18 and 22 carbon atoms. The monocarboxylic acid may be selected from the group comprising lauric acid, myristic acid, palmitic acid, stearic acid, arachidic acid, and behenic acid. Preferably, the monocarboxylic acid may be selected from stearic acid and behenic acid.

Fatty acids suitable for use herein can be obtained from natural sources such as, for example plant, or animal esters. For example, the acids may be obtained from palm oil, rape seed oil, palm kernel oil, coconut oil, babassu oil, soybean oil, castor oil, sunflower oil, olive oil, linseed oil, cottonseed oil, safflower oil, tallow, whale or fish oils, grease, lard and mixtures thereof. The fatty acids can also be synthetically prepared. Relatively pure unsaturated fatty acids such as oleic acid, linoleic acid, linolenic acid, palmitoleic acid, and elaidic acid may be isolated, or relatively crude unsaturated fatty acid mixtures employed. Resin acids, such as those present in tall oil, may also be used.

Preferably, the monocarboxylic acid is saturated. The fatty acid may be either a branched fatty acid or a linear fatty acid. A mixture of fatty acids may be present. In this case, the mixture may comprise branched fatty acids, linear fatty acids, or a mixture thereof.

Preferably, the molar ratio of the end-cap to the reaction product of the alkoxylated polyol or ester thereof and the dicarboxylic acid is at least 0.05:1, preferably at least 0.1:1, more preferably at least 0.2:1, and most preferably at least 0.35:1. Preferably, molar ratio of the end-cap to the reaction product of the alkoxylated polyol or ester thereof and the dicarboxylic acid is up to 20:1, preferably up to 10:1, more preferably up to 5:1, and most preferably up to 2:1.

Preferably, the molar ratio of the alkoxylated polyol or ester thereof to the dicarboxylic acid to the end-cap is at least 1:0.1:1, and more preferably at least 1:0.5:1. Preferably, the molar ratio of the end-cap to the reaction product of the alkoxylated polyol or ester thereof and the dicarboxylic acid is up to 1:1:5, and more preferably up to 1:1:2.

Preferably, the reaction product has a molecular weight of greater than 700 Daltons, preferably greater than 1,000 Daltons, more preferably greater than 1,500 Daltons, and most preferably greater than 2,000 Daltons. Preferably, the reaction product has a molecular weight of less than 100,000 Daltons, preferably less than 80,000 Daltons, more preferably less than 50,000 Daltons, and most preferably less than 20,000 Daltons.

Preferably, the reaction product has a toxicity of less than 20,000 mg/1, more preferably less than 15,000 mg/1, further preferably less than 10,000 mg/1, and most preferably less than 5,500 mg/l. The toxicity is determined according to the method in ISO 10253 (Second edition, 15.04.2006).

Preferably, the reaction product has a viscosity at 25° C. of greater than 100 mPa·s, more preferably greater than 300 mPa·s, further preferably greater than 500 mPa·s, and most preferably greater than 900 mPa·s. The viscosity is measured at 25° C. on a Brookfield viscometer using a 29 Spindle at a shear rate of 0.25N.

Preferably, the reaction product has a pour point of less than 100° C., more preferably less than 80° C., further preferably less than 50° C., and most preferably less than 30° C. Preferably, the reaction product has a pour point of greater than 1° C., more preferably greater than 5° C., further preferably greater than 10° C. Preferably, the pour point is measured on an ISL MPP 5Gs automated pour point analyser according to the ASTM D97 standard method.

Preferably, the reaction product has a pH which is approximately neutral. Preferably, the reaction product has a pH of between 3 and 12, more preferably between 4 and 10, further preferably between 5 and 8, and most preferably between 6 and 7. The pH of the reaction product is measured at a concentration of 1% in an 85% IPA solution using an HI 8424 portable pH probe.

Preferably, the reaction product has a density at 25° C. of at least 0.1 g/cm³, more preferably at least 0.5 g/cm³, further preferably at least 0.8 g/cm³, and most preferably at least 1.0 g/cm³. Preferably, the reaction product has a density at 25° C. of up to 10 g/cm³, more preferably up to 5 g/cm³, further preferably up to 3 g/cm³, and most preferably up to 2 g/cm³. The density may be determined by pouring 10 ml of sample into a measuring cylinder and calculating the approximate density from the weight.

Preferably, the reaction product has good thermal stability in air and/or nitrogen. Preferably, the reaction product is stable in air up to a temperature of at least 50° C., more preferably at least 100° C., further preferably at least 150° C., and most preferably at least 200° C. before the product starts to degrade. Preferably, the reaction product is stable in nitrogen up to a temperature of at least 50° C., preferably at least 100° C., more preferably at least 150° C., and most preferably at least 200° C. before the product starts to degrade. The thermal stability in air tests were performed at 150° C. and 200° C. The experimental method was as follows:
Thermogravimetric Analysis (TGA) at 150° C. over 1 hour—Between 10 and 15 mg of the sample to be tested was weighed into a 70 μL alumina crucible put into the thermogravimetric analyser's (Mettler TG50) furnace and run under air with
   Gas (flow rate): Air (200 ml/min)
   Temperature range: 30-150° C. at 50° C./min then, 150° C. for one hour then, 150-600° C. at 50° C./min then, 600° C. for five minutes.

For TGA Analysis at 200° C. over 1 hour—The method described for TGA Analysis at 200° C. was performed, but over the temperature range: 30-200° C. at 50° C./min, then 200° C. for one hour, then 200-600° C. at 50° C./min, then 600° C. for five minutes.

Preferably, the reaction product shows a mass loss in air over a period of 1 hour at 150° C. of less than 50%, preferably less than 30%, more preferably less than 15%, and most preferably of less than 7%. Preferably, the reaction product shows a mass loss in air over a period of 1 hour at 200° C. of less than 90%, preferably less than 85%, more preferably less than 80%, and most preferably of less than 75%.

The percentage mass loss was calculated by step horizontal analysis using STARe software (version 9.2) on the results of the TGA at 150° C. and TGA at 200° C. methods described herein.

The cleaning formulation comprises the reaction product of an alkoxylated polyol or ester thereof and a dicarboxylic acid.

The cleaning formulation may comprise said reaction product in the range from 0.01 wt. % to 40 wt. %. Preferably, in the range from 0.05 wt. % to 30 wt. %. More preferably, in the range from 0.07 wt. % to 20 wt. %. Further preferably, in the range from 0.1 wt. % to 15 wt. %. Most preferably, in the range from 0.1 wt. % to 10 wt. %.

The cleaning formulation may be formed by diluting the cleaning composition, where said dilution may be achieved either by addition of a solvent such as water, or by addition of the cleaning composition to an existing cleaning formulation in order to provide soil resistance to said existing formulation.

Whilst the above amounts refer to the actual amount of the reaction product in the cleaning formulation, it is envisaged that the components may be added to the formulation in the form of dilute solutions.

The formulation containing the reaction product of an alkoxylated polyol or ester thereof and a dicarboxylic acid may be used solely or mainly to provide treatment of the hard surfaces in order to improve both soil repellency and soil removal. The cleaning formulation may therefore be an end-use cleaning formulation. Said formulation will typically be formulated in water and contain said reaction product.

In cleaning formulations other components may include surfactants including detergents, wetters and/or dispersants; soil suspending agents and/or anti-redeposition agents; dye transfer agents and/or dye transfer inhibitors; enzymes; bleaches optionally with bleach activators; hydrotropes; builders; sequestrants (chelating agents); pH adjustment and/or buffering agents; solids such as mild abrasives; corrosion inhibitors; anti-foams; stabilisers; preservatives, particularly biocides such as anti-microbials; radical scavengers; perfume; anti dusting agents; optical brighteners; silicones; dye/pigment; all typically at conventional levels.

It should be understood that by the term ‘soils’ it is included materials which would otherwise adhere to a hard surface or be difficult to remove therefrom. Soils may include those made substantially of mineral deposits, such as alkali metal, particularly calcium and/or magnesium carbonates, and stains which include such mineral deposits combined with other soil such as water insoluble soap salts, such as calcium and/or magnesium stearates. Soils may also include, but are not limited to, particulate/oily type soils, grease (animal or vegetable), and exterior window soils. Other soils are described herein.

As applied to hard surfaces in accordance with the second aspect, the invention will typically comprise the following steps:
a) applying to a hard surface an aqueous cleaning composition in accordance with the first aspect or cleaning formulation accordance with the third aspect of the present invention, typically by pouring or spraying the composition or formulation on the hard surface or by other application means;
b) simultaneously or subsequently spreading and/or wiping the composition or formulation over the hard surface, usually with a fibrous or porous wiping or spreading means; and then
c) optionally removing or rinsing at least part of the liquid, from the hard surface with water and/or a fibrous or porous drying means, some dirt being removed from the hard surface in the rinse water and/or the drying means.

It has been found that when a composition of the first aspect is present in cleaning formulations, it covers and adheres to hard surfaces being cleaned and modifies the surface giving soil repellency. This reduces soil material build-up on the surface to start with, and also makes it easier to rinse off soils from the surface.

All of the features described herein may be combined with any of the above aspects, in any combination.

In order that the present invention may be more readily understood, reference will now be made, by way of example, to the following description.

It will be understood that all tests and physical properties listed have been determined at atmospheric pressure and room temperature (i.e., 20° C.), unless otherwise stated herein, or unless otherwise stated in the referenced test methods and procedures.

### Synthesis of Hard Surface Cleaning Compound (H1)

A composition comprising sorbitan (20EO) monolaurate and adipic acid in a 2:1 molar ratio was produced.

384 kg of sorbitan (20EO) monolaurate was added to the reaction vessel and heated to 80° C. whilst stirring. 23 kg of adipic acid flake was slowly added to the warm stirred sorbitan (20EO) monolaurate. The mixture was heated to 235° C. observing distillation water removal. The reaction was continued until an acid value of the less than 5 mg KOH/g was observed.

The reaction yielded 400 kg of reaction product and 5.8 kg water.

The reaction product was analysed and the physical properties measured are shown below.

Appearance—Amber viscous liquid

Viscosity 25° C. Brookfield Spindle 29—970 cP

Density 25° C.—1.0552 g/cm³

Cloud Point 5% sample in 35% BDG: 65% DI Water—+82° C.

Pour Point, Pour Point Analyser—+24° C.

pH 1% Sample in 85:15 IPA:DI Water—6.1

Mass lost at 150° C.—6.6%

Mass lost at 200° C.—55.7%

The toxicity of H1 was tested by the methods described herein. The results are given below.

Toxicity—238 mg/l

It can be seen that H1 has low toxicity properties.

### Example Cleaning Formulations

Cleaning compositions comprising H1 were formulated and used on a number of different surfaces to test the effectiveness as a hard surface cleaner.

### Example 1

### Ceramic Tile with Soap Scum

A modified version of CSPA method DCC-16 “Guidelines for Evaluating the Efficacy of Bathroom Cleaners” was used to test H1 on ceramic tiles. The ceramic tiles were first cleaned with a test solution and left to dry, and then the tiles were treated by spraying with a lab made soap scum solution.

A parent soil was prepared, and the composition is shown in Table 1.

TABLE 1
Parent soil composition

| Component | Amount (wt. %) |
|---|---|
| Bar Soap | 3.90 |
| Shampoo | 0.35 |
| Clay | 0.06 |
| Artificial Sebum | 0.15 |
| Hard Water | 95.54 |

The parent soil composition was then added to a final soap scum soil, the composition of which is shown in Table 2.

TABLE 2
Final soap scum soil composition

| Component | Amount (wt. %) |
|---|---|
| Acetone | 85.72 |
| Parent Soil* | 4.5 |
| Carbon Black | 0.01 |
| 0.1N HCl | 0.77 |
| Hard Water (20,000 ppm) | 9.0 |

The resulting final soap scum soil composition was formulated to measure cleaning efficacy of particular cleaning formulations and to show effectiveness of H1. The cleaning formulations used were:
C1—H1 at 1% in water
C2—Acid cleaner with 1% H1
C3—Acid cleaner

White ceramic tiles were then sprayed with the test solutions C1 to C3. An untreated tile was also tested as a negative control.

After treatment, tiles were sprayed with the prepared soil above evenly coating each tile. The soiled tiles were baked in an 85° C. oven for 15 minutes to secure the soil. The tiles were removed and allowed to cool to room temperature.

A Gardner scrub apparatus was used to clean each tile. Soiled tiles were secured to the apparatus and subject to a damp sponge secured in a weighted holder moving across the tile surface. The sponge was dampened with water and sprayed with a cleaner before passing over each tile for 5 strokes. Tiles were removed and evaluated on a Gardner colorimeter to determine soil removal. The results are shown in Table 3.

TABLE 3
Soil removal from ceramic tile

| Cleaner Formulation | Soil Removal (%) |
|---|---|
| C1 | 99.4 |
| C2 | 98.1 |
| C3 | 10 |
| Control | 10 |

As shown in Table 3, H1 performed well, and greatly improved a cleaner that has minimal efficacy as a cleaner for this soil/substrate combination.

### Example 2

### Glass Surface with Exterior Soil

A method similar to CSPA DCC-09 “Glass Cleaners” was used to measure the surface modification properties of H1 on a glass surface. Glass slides were sprayed with the test solutions shown in the list below.

### Test Solutions:

C4—H1 at 1.0% in water
C5—H1 at 0.5% in water
C6—Cleaner (Windex®) with 0.5% H1
C7—Cleaner (Windex®) (Control)

After treatment with the test solution the panels were allowed to dry before they were sprayed with exterior soil as specified in the DCC-09 method listed above. The composition of the exterior soil is shown in Table 4.

TABLE 4
Exterior soil composition

| Cleaner Formulation | Amount (%) |
|---|---|
| Mineral oil | 0.75 |
| Clay | 0.75 |
| Acetone | 98.5 |

After the glass slides were soiled and dried they were rinsed with approximately 25-30 ml de-ionised water at room temperature. Slides were measured for percentage transmittance using a Genesys 6 UV-visible spectrophotometer which was used to calculate the soil removed (% SR) by the following equation.

$$Glass slide ( as related to trasnmittance ) \% SR = \frac{{T}_{t} - {T}_{s}}{{T}_{o} - {T}_{s}} \times 100$$

where:
Tₛ=transmission of soiled slide
Tₒ=transmission of original slide
Tₜ=transmission of treated or rinsed slide

A glass slide treated with cleaner only (C7) was used as a negative control. The results are shown in Table 5.

TABLE 5
Soil removal from glass surface

| Cleaner Composition | Soil Removal (%) |
|---|---|
| C4 | 100 |
| C5 | 97 |
| C6 | 98 |
| C7 (Control) | 3 |

As shown in Table 5, the H1 containing formulations provided improved soil removal performance at inclusion at 1.0 and 0.5% (C4 and C5). Additionally, addition of H1 at levels of 0.5% to an existing commercially available cleaner (C6) provides similarly improved soil removal performance. This is in contrast to the poor soil removal shown by the control cleaner in which no H1 was included (C7).

It was also found that H1 provides good anti-fog efficacy when added to a commercial window cleaner, whilst without addition of H1 no anti-fog effect were seen.

### Example 3

### Pre-Painted Steel Panels with Lard

Another substrate and soil combination was selected to test the efficacy of H1. The surface used was a painted steel panel subjected to a lard/carbon black soil. The metal panels were pre-painted cold rolled steel metal panels, similar to a stove top or refrigerator surface.

Each panel was treated with a surface modifier by spraying the agent on the panel and letting it dry. The agents tested are shown below.
C8—H1 at 1%
C9—Alkaline cleaner with 1% H1

An untreated panel was used as a negative control.

After surface treatment, a lard/carbon black soil was applied by using a pull down bar at 0.8 mil thickness. Each panel was subject to a scrub test using a damp sponge sprayed with a dilute cleaner. A low grade cleaner was intentionally chosen to see the effects of the surface modification agent and not the cleaner. The test consisted of 4 cycles of scrubbing using the Gardner scrub apparatus. Soil removal was measured on a Gardner colorimeter apparatus using the L value, as described with regard to Example 4. The results are shown in Table 6.

TABLE 6
Soil removal from steel surface

| Cleaner Composition | Soil Removal (%) |
|---|---|
| Control | 49 |
| C8 | 96 |
| C9 | 87 |

As shown in Table 6, H1 (C8) removed just about all the soil from the surface.

### Example 4

### Vinyl Tiles with Particulate Soil

A modified version of “Standard Guide for Testing Cleaning Performance of Products Intended for Use on Resilient Flooring and Washable Walls” ASTM D4488-85 was used to test H1 on vinyl tiles. Standard vinyl tiles were sprayed with test solutions (shown below) and allowed to dry.
C10—H1 at 1%
C11—Neutral cleaner with 1% H1
C12—Neutral cleaner

A vinyl tile with no treatment was included as a negative control.

After treatment the tiles were tested according to the Gardner Scrub Test. Testing was performed using linear motion washability and wear test equipment from Gardner Co.

Soiled tiles were prepared by spreading a prepared soil (shown in Table 7) on a vinyl tile, allowing it to air dry, followed by heating in an oven at 80° C. for 20 minutes.

TABLE 7
Prepared soil composition

| Ingredient | Amount (wt. %) |
|---|---|
| Odourless mineral spirits | 65.10 |
| Carnation mineral oil | 1.70 |
| Crisco ® | 1.60 |
| Carbon black | 0.30 |
| Metallic brown iron oxide | 15.00 |
| Black charm clay | 15.00 |
| Used motor oil | 1.30 |

In one set of tests a damp sponge with only tap water was used to scrub the soiled tiles, while in the second set of tests the sponge was sprayed with a neutral type cleaner. The machine was started and the sponge holder cycled back and forth 2 times for the water only run, and 1 time for the neutral cleaner type. The tiles were removed and rinsed under a light stream of cold tap water for a couple of seconds.

The slides were examined on the Gardner colour instrument to measure percentage soil removal (% SR) using the L value (black to white). This measurement was calculated from the reflectance of the cleaned coupon, soiled coupon and the original.

$$\% SR = \frac{{L}_{t} - {L}_{s}}{{L}_{o} - {L}_{s}} \times 100$$

where:
Lₒ is the original unsoiled L value for the tile
Lₛ is the soiled L value for the tile
Lₜ is the L value for the treated or washed tile

The results are shown in Table 8.

TABLE 8
Soil removal from vinyl tile surface

Soil Removal (%)

| Cleaner | | Clean with neutral |
|---|---|---|
| Composition | Clean with water | cleaner |
| Control | 45 | 60 |
| C10 | 64 | 79 |
| C11 | 56 | 69 |
| C12 | 44 | 56 |

As shown in Table 8, C10 had the best cleaning and soil removal performance. Also, H1 added to a neutral cleaner (C11) enhanced the performance compared to the same cleaner without H1 added (C12). Even when using only a damp sponge, the pre-treated H1 tiles showed substantial soil removal.

### Example 5

### Painted Metal Surface with Motor Oil

A test which used a painted metal surface soiled with used motor oil was undertaken. A painted metal panel was treated by spraying and wiping on a 1% solution of H1. Used motor oil was then spread over the treated surface. An untreated panel (control) was similarly soiled with used motor oil. Both soiled panels were immersed in plain tap water at −30° C.

It was observed that substantially all the motor oil on the treated panel came off in under 30 seconds when the panel was immersed in water. The resulting treated surface was visibly free from all oily soil. In contrast, the motor oil on the untreated control panel remained unchanged and did not come off upon immersion.

As this particular test involved no mechanical activity, minimal temperature, and minimal time, the chemical contribution provided by the use of H1 was determined to be the main factor in soil removal.

It is to be understood that the invention is not to be limited to the details of the above embodiments, which are described by way of example only. Many variations are possible.

1. A cleaning composition comprising the reaction product of:
a) an alkoxylated polyol or ester thereof; and
b) a dicarboxylic acid.

2. The cleaning composition according to claim 1, wherein the polyol in the alkoxylated species comprises at least 3 hydroxyl groups.

3. The cleaning composition according to claim 1, wherein the polyol in the alkoxylated species has the general formula (Ia):
HOH₂C—(CHOH)ₚ—CH₂OH (Ia)
where p is from 1 to 6.

4. The cleaning composition according to claim 1, wherein the alkoxylated polyol comprises residues of a univalent radical R²—O—, or anion R²—O⁻, where R² is an alkyl group.

5. The cleaning composition according to claim 1, wherein the alkoxylated polyol or ester thereof comprises between 1 and 500 alkylene oxide equivalents per molecule.

6. The cleaning composition according to claim 1, wherein the dicarboxylic acid has the general formula (IV):
HOOC—R⁴—COOH (IV)
where R⁴ is a C₂ to C₃₆ hydrocarbyl group which is saturated or unsaturated, linear or branched, and aromatic or aliphatic.

7. The cleaning composition according to claim 1, wherein the molar ratio of alkoxylated polyol or ester thereof to dicarboxylic acid in the reaction product is at least 0.05:1 and up to 20:1.

8. The cleaning composition according to claim 1, wherein the reaction product of the alkoxylated polyol or ester thereof and the dicarboxylic acid further comprises an end-cap.

9. The cleaning composition according to claim 8, wherein the end-cap comprises a monocarboxylic acid.

10. The cleaning composition according to claim 8, wherein the molar ratio of the end-cap to the reaction product of the alkoxylated polyol or ester thereof and the dicarboxylic acid is at least 0.05:1 and up to 20:1.

11. The cleaning composition according to claim 8, wherein the molar ratio of the alkoxylated polyol or ester thereof to the dicarboxylic acid to the end-cap is at least 1:0.1:1 and up to 1:1:5.

12. A cleaning formulation, said formulation comprising in the range from 0.01 wt. % to 40 wt. % of the cleaning composition of claim 1.

13. A method of treating a hard surface which comprises applying to the surface a cleaning composition, said composition comprising the reaction product of:
a) an alkoxylated polyol or ester thereof; and
b) a dicarboxylic acid.

14. A method for providing soil resistance to a hard surface, comprising applying to the hard surface the cleaning composition according to claim 1.
